SEQUENCE LISTING (3 sequences)

SEQ ID NO: 1 - protein, 14 aa, Artificial Sequence
  Synthetic peptide.
LAQCFFCFKELEGW

SEQ ID NO: 2 - protein, 15 aa, Artificial Sequence
  Synthetic peptide.
EFYLAMPFATPMEAE

SEQ ID NO: 3 - protein, 15 aa, Artificial Sequence
  Synthetic peptide.
NTEMFVTAPDNLGYT

(19) United States
(12) Patent Application Publication
(10) Pub. No.: US 2012/0027674 A1
(43) Pub. Date: Feb. 2, 2012

(54) IL-18 RECEPTOR AS A NOVEL TARGET OF REGULATORY T CELLS IN CANCER

(75) Inventors: Hideki Ueno, Plano, TX (US); Jacques F. Banchereau, Montclair, NJ (US); Anna Karolina Palucka, Dallas, TX (US)

(73) Assignee: BAYLOR RESEARCH INSTITUTE, Dallas, TX (US)

(21) Appl. No.: 13/187,843

(22) Filed: Jul. 21, 2011

Related U.S. Application Data
(60) Provisional application No. 61/368,174, filed on Jul. 27, 2010.

(51) Int. Cl. A61K 51/00; A61K 38/16; A61P 37/04; A61K 38/00; A61P 35/00; A61K 39/00; A61K 39/395; A61K 38/47

(52) U.S. Cl. 424/1.11; 424/130.1; 424/133.1; 514/21.2; 424/94.61; 514/2.1; 424/184.1

(57) ABSTRACT

Compositions and methods for use in preventing, inhibiting or reducing tumor cell growth comprising an effective amount of an active agent that kills IL-18 Receptor expressing T cells in admixture with a suitable diluent or carrier are described herein.

### CROSS-REFERENCE TO RELATED APPLICATIONS

This patent application is a non-provisional application of U.S. Provisional Patent Application No. 61/368,174 filed on Jul. 27, 2010 and entitled “IL-18 Receptor as A Novel Target of Regulatory T Cells in Cancer” which is hereby incorporated by reference in its entirety.

### STATEMENT OF FEDERALLY FUNDED RESEARCH

This invention was made with U.S. Government support under Contract No. P0-1 CA84512 awarded by the NIH. The government has certain rights in this invention.

### TECHNICAL FIELD OF THE INVENTION

The present invention relates in general to the field of T cell regulation, and more particularly, to composition for the treatment of cancer by eliminating or reducing the effect of Regulatory T Cells.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with the generation and uses for Regulatory T cells.

U.S. Pat. No. 7,651,855, issued to Blazar, et al. (2010), is directed to Regulatory T cells (Treg cells) and their use in immunotherapy and suppression of autoimmune responses. Briefly, this patent is said to teach methods for the production of ex vivo activated and culture-expanded isolated CD4⁺CD25⁺ suppressor Treg cells for the prevention or suppression of immune reactions and autoimmune responses in a host, e.g., a human host. These ex vivo, culture-expanded Treg cells are also said to provide a sufficient amount of otherwise low numbers of such cells, having long term suppressor capability to permit therapeutic uses, including the preventing, suppressing, blocking or inhibiting the rejection of transplanted tissue in a human or other animal host, or protecting against graft versus host disease.

U.S. Pat. No. 7,115,259, issued to Horwitz (2006) is directed to the use of cytokines and mitogens to inhibit pathological immune responses. Briefly, the invention is said to be related to methods of treating autoimmune diseases, including both antibody-mediated and cell-mediated disorders. The invention includes a method for treating an autoimmune disorder in a patient by removing peripheral blood mononuclear cells (PBMC) from the patient, treating the cells with a regulatory composition to generate regulatory T cells, said regulatory composition comprising anti-CD2 and anti-CD3, and reintroducing said regulatory T cells to the patient to suppress an aberrant immune response.

U.S. Patent Application Publication No. 2010092488, filed by Suzumura, et al. (2010), is directed to methods for the treatment or prevention of diseases associated with functional disorders of regulatory T Cells. Briefly, these inventors examined the role of midkine (MK) in experimental autoimmune encephalomyelitis; a human model for multiple sclerosis. MK is said to inhibit regulatory T cells and that the autoimmune mechanism induced by type 1 helper T cells can be suppressed by inhibiting MK expression or its activity, thereby increasing the number of regulatory T cells. Diseases associated with the functional disorder of regulatory T cells are said to be treated with the administration of an inhibitor that inhibits MK expression or activity.

Finally, U.S. Patent Application Publication No. 2010003287, filed by Mills, et al. (2010), is directed to compositions and methods relating to treatment of cancer and infectious diseases in which an immune response is regulated by the administration of a composition comprising a Toll-like receptor agonist and an immune mediator which downregulates the expression of the anti-inflammatory cytokine IL-10 and upregulates the expression of the pro-inflammatory cytokine IL-12. The application is said to include methods that can be used to provide therapeutic treatment for cancerous conditions and infectious diseases.

### SUMMARY OF THE INVENTION

The present invention in various embodiments describes uses of compositions and methods for preventing, inhibiting or reducing tumor cell growth. The composition of the present invention comprises an effective amount of an active agent that kills IL-18 Receptor expressing T cells in admixture with a suitable diluent or carrier.

The present invention provides for a method of preventing, inhibiting or reducing tumor cell growth comprising administering an effective amount of an active agent comprising an IL-18 Receptor (IL-18R) binding molecule to a cell or an animal in need thereof sufficient to kill tumor specific regulatory T cells. The method of the present invention further comprises the step of injecting the patient with a tumor cell-specific dendritic cell vaccine. In one aspect the active agent comprises an anti-IL-18R antibody or fragments thereof, an IL-18R antagonist, an IL-18 Fc, or an IL-18 Toxin. In another aspect of the method of the present invention the antibody comprises an Fab, Fv, scFv, Fab′ and F(ab′)₂ antibody fragment, wherein the antibody is a humanized antibody. In yet another aspect the antibody is an immunotoxin that comprises an IL-18 Receptor antigen binding fragment and a cytotoxic agent selected from the group consisting of toxins, antibiotics, radioactive isotopes and nucleolytic enzymes.

In specific aspects the cytotoxic agent is (i) a toxin selected from the group consisting of auristatin; maytansinoid; calicheamicin; mono-methyl auristatin E; mono-methyl auristatin F; auristatin E valeryl benzylhydrazone; and Auristatin F phenylene diamine, (ii) is from the group consisting of ricin; abrin; alpha toxin; saporin; ribonuclease (RNase); deoxyribonuclease; Staphylococcal enterotoxin-A; pokeweed antiviral protein; gelonin; diphtheria toxin; Pseudomonas exotoxin; and Pseudomonas endotoxin, (iii) is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi, and (iv) is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

In one aspect the active agent further comprises an antitumor drug. In another aspect the T cells are IL-18R^{high}, FoxP3+, CXCR3+. In yet another aspect the T cells express at least one of CTLA-4, CD25, CD28, and T-bet. In a related aspect the tumor is at least one of a melanoma cancer; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors; and squamous cell carcinomas.

In another embodiment the present invention relates to a pharmaceutical composition for use in preventing, inhibiting or reducing tumor cell growth, comprising an effective amount of an active agent that kills IL-18 Receptor expressing T cells in admixture with a suitable diluent or carrier. In one aspect the composition further comprises the step of injecting the patient with a tumor cell-specific dendritic cell vaccine. In another aspect the active agent comprises an anti-IL-18R antibody or fragments thereof, an IL-18R antagonist, an IL-18 Fc, or an IL-18 Toxin. In yet another aspect the T cells are Regulator T cells that are FoxP3+, CXCR3+. In another aspect the antibody comprises an Fab, Fv, scFv, Fab′ and F(ab′)₂ antibody fragment. In a specific aspect the antibody is a humanized antibody.

In one aspect the antibody is an immunotoxin that comprises an IL-18 Receptor antigen binding fragment and a cytotoxic agent selected from the group consisting of toxins, antibiotics, radioactive isotopes, and nucleolytic enzymes. In another aspect the cytotoxic agent is a toxin selected from the group consisting of auristatin; maytansinoid; calicheamicin; mono-methyl auristatin E; mono-methyl auristatin F; auristatin E valeryl benzylhydrazone; and Auristatin F phenylene diamine. In another aspect the cytotoxic agent is from the group consisting of ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin. In another aspect the cytotoxic agent is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi. In another aspect the cytotoxic agent is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs. In yet another aspect the active agent further comprises an antitumor drug. In a related aspect the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors, and squamous cell carcinomas.

Yet another embodiment of the instant invention provides a method of reducing tumor cell growth or metastasis comprising administering an active agent that kills IL-18 R^{high} Regulatory T cells. In a specific aspect T cells are IL-18R^{high}, FoxP3+, CXCR3+. In one aspect the T cells express at least one of CTLA-4, CD25, CD28, and T-bet.

One embodiment of the present invention discloses a method comprising screening cancer patients to identify those in which expression of IL-18R is upregulated on T cells and administering an active agent that kills the T cells. The method further comprises the step of injecting the patient with a tumor cell-specific dendritic cell vaccine. In one aspect the active agent comprises an anti-IL-18R antibody or fragments thereof, an IL-18R antagonist, an IL-18 Fc, or an IL-18 Toxin. In a specific aspect the antibody comprises an immunotoxin. In another aspect the antibody comprises an Fab, Fv, scFv, Fab′ and F(ab′)₂ antibody fragment. In yet another aspect the antibody is a humanized antibody.

In another aspect the immunotoxin comprises an IL-18 Receptor antigen binding fragment and a cytotoxic agent selected from the group consisting of toxins, antibiotics, radioactive isotopes, and nucleolytic enzymes. In another aspect the cytotoxic agent is a toxin selected from the group consisting of auristatin; maytansinoid; calicheamicin; mono-methyl auristatin E; mono-methyl auristatin F; auristatin E valeryl benzylhydrazone; and Auristatin F phenylene diamine. In another aspect the active agent is from the group consisting of ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin. In another aspect the active agent is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi. In another aspect the active agent is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

In yet another aspect the active agent further comprises an antitumor drug. In a related aspect the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors, and squamous cell carcinomas.

The instant invention in another embodiment discloses a method for determining whether a tumor will respond to anti-IL-18 Receptor therapy comprising: isolating T cells from a subject suspected of having a tumor and determining the level of expression of IL-18R on the T cells isolated from the subject, wherein an increase in the level of IL-18R on T cells as compared to a normal subject is indicative that the patient will respond to anti-Regulatory T cell therapy.

The method described hereinabove further comprises the step of injecting the patient with a tumor cell-specific dendritic cell vaccine. In one aspect the T cells are also evaluated for the expression of at least one of CTLA-4, CD25, CD28, FoxP3, CXCR3, and T-bet. The method as described hereinabove further comprises the steps of treating the subject with an anti-IL-18 Receptor antibody or fragments thereof, an IL-18R antagonist, an IL-18 Fc, or an IL-18 Toxin and the step of treating the patient with an antitumor drug.

In one aspect the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors, and squamous cell carcinomas. In another aspect the T cells are IL-18R^{high}, FoxP3+, and CXCR3+. In yet another aspect T cells express at least one of CTLA-4, CD25, CD28, and T-bet.

Yet another embodiment relates to a method of treating melanoma comprising administering an effective amount of an active agent comprises an IL-18 Receptor (IL-18R) binding molecule to a cell or an animal in need thereof sufficient to kill specific regulatory T cells in the melanoma. The method further comprises the step of injecting the patient with a melanoma-specific dendritic cell vaccine.

Finally, a pharmaceutical composition for use in inhibiting or reducing melanoma cell growth comprising an effective amount of an active agent that kills IL-18 Receptor expressing T cells in the melanoma in an admixture with a suitable diluent or carrier is disclosed herein. In one aspect the composition further comprises the step of injecting the patient with a tumor cell-specific dendritic cell vaccine.

### BRIEF DESCRIPTION OF THE DRAWINGS

For a more complete understanding of the features and advantages of the present invention, reference is now made to the detailed description of the invention along with the accompanying figures and in which:

FIG. 1: IL-10⁺ melanoma-specific T reg cell lines express higher levels of IL-18R1 and IL-18R2 transcripts;

FIG. 2: Activated blood melanoma-specific T regs express IL-18Rα. PBMCs from a melanoma patient were cultured for 7 d with a T reg epitope peptide (NY-ESO-1 derived 15mer). Expression of IL-18Rα and FoxP3 in CD4+ T cells was analyzed by flow cytometry;

FIG. 3: Blood CD4+CD25+ T regs from metastatic melanoma patients upregulate IFN-γ secretion in response to IL-18;

FIG. 4: T regs found in metastatic melanoma tumors, T regs co-expressed FoxP3, CD25, and CTLA4;

FIG. 5: T regs found in metastatic melanoma tumors express high levels of IL-18Ra. Gated to CD45+CD3+CD4+ T cells;

FIG. 6: IL-18Rα is expressed by T regs at highest levels in metastatic melanoma tumors. IL-18Rα expression level was analyzed on CD4+ T cell subsets (left), CD8+ T cell subsets (middle), and non-T cell lymphocyte populations (right) infiltrating into a metastatic melanoma tumor;

FIG. 7: FoxP3+ T regs found in metastatic melanoma tumors express CXCR3. Gated to CD45+CD3+CD4+ T cells;

FIG. 8: IL-18R+FoxP3+ T regs express T-bet. Gated to CD4+CD3+CD45+ T cells;

FIG. 9 is a schematic showing the mechanism by which CXCR3+ T regs suppress anti-tumor immunity at tumor sites mediated by NK cells and CD8+ T cells; and

FIG. 10 is a plot showing that IL-18R1 is dominantly expressed by plasmacytoid DCs (pDCs), NK cells, and T cells in humans.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes compositions and methods for use in preventing, inhibiting or reducing tumor cell growth by targeting an effective amount of an active agent that kills IL-18 Receptor expressing Regulatory T cells in admixture with a suitable diluent or carrier is described herein, rather than attempting to control the regulatory T cell activity.

The compositions and method taught herein were used to inhibit or reduce tumor cell growth by administering an effective amount of an active agent (e.g., an anti-IL-18R antibody or fragments thereof, an IL-18R antagonist, an IL-18 Fc or an IL-18 Toxin) to kill IL-18 Receptor expressing T cells. This causes a depletion or suppression of the tumor-infiltrating T regs (which if left unchecked would otherwise suppress the anti-tumor immunity mediated by NK cells and CD8+ T). The findings of the present invention on T-reg suppression serve as an alternative novel approach to tumor treatment.⁷⁻⁸ Colombo and Piconese, have discussed the targeting of antigen specific Regulatory T-cell inhibition as the right choice in cancer immunotherapy because depletion would have the opposite effect, which might cause de novo induction of Regulatory T-cells. Nature Reviews Cancer 7, pp. 880-887 (2007).

An IL-18R binding molecule can include a protein such as IL-18 Toxin molecule, an IL-18 molecule fused to the constant region of an antibody (IL-18 Fc) that include the portions of the antibody that lead to complement fixation by the host, an antibody that binds the IL-18R and that triggers Complement fixation and cell death, immunotoxins specific for the IL-18R and even proteins, lectins, and small molecules that bind specifically to IL-18R expressing Regulatory T cells in and around a tumor or cancer leading to death of the Regulatory T cells.

As used herein, the term “IL-18R binding antibody” or “anti-IL-18R antibody” refers to any antibody capable of binding to the IL-18R antigen. The binding reaction may be shown by standard methods (qualitative assays) including, for example, a bioassay for determining by blocking the binding of other molecules to IL-18R or any kind of binding or activity assays (e.g., activation, reduction or modulation of an immune response), with reference to a negative control test in which an antibody of unrelated specificity but often of the same isotype, e.g., an anti-CD25 or anti-CD80 antibody, is used. In some cases the level of expression of IL-18R is measured in a patient having a cancer or tumor as compared to a normal patient, e.g., a subject that does not have a cancer or tumor and that will often be matched by, e.g., age, gender, race, medical history, or location, as is commonly used by medical professionals.

As used herein, the term “chimeric antibody” refers to an antibody in which the constant regions of heavy or light chains or both are of human origin while the variable domains of both heavy and light chains are of non-human (e.g., mouse, hamster, or rat) origin, or of human origin but derived from a different human antibody.

As used herein, the term “CDR-grafted antibody” refers to an antibody in which the hypervariable complementarity determining regions (CDRs) are derived from a donor antibody, such as a non-human (e.g., mouse) antibody, or a different human antibody, while all or substantially all the other parts of the immunoglobulin (e.g., the conserved regions of the variable domains, i.e., framework regions), are derived from an acceptor antibody (in the case of a humanized antibody—an antibody of human origin). A CDR-grafted antibody may include a few amino acids of the donor sequence in the framework regions, for instance in the parts of the framework regions adjacent to the hypervariable regions.

As used herein, the term “human antibody” or “humanized” antibody refers to an antibody in which the constant and variable regions of both the heavy and light chains are all of human origin, or substantially identical to sequences of human origin, not necessarily from the same antibody and includes antibodies produced by mice in which the mouse, hamster or rat immunoglobulin variable and constant part genes have been replaced by their human counterparts, e.g. as described in general terms in EP 0546073 B1, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, EP 0 438474 B1 and EP 0 463151 B1, relevant portions incorporated herein by reference. The constant region domains preferably also comprise suitable human constant region domains, for instance as described in “Sequences of Proteins of Immunological Interest”, Kabat et al. U.S. Department of Health and Human Services, Public Health Service, and National Institute of Health.

As used herein, the term “veneered” refers to a humanized antibody framework onto which antigen-binding sites or CDRs obtained from non-human antibodies (e.g., mouse, rat or hamster), are placed into human heavy and light chain conserved structural framework regions (FRs), for example, in a light chain or heavy chain polynucleotide to “graft” the specificity of the non-human antibody into a human framework. The polynucleotide expression vector or vectors that express the veneered antibodies can be transfected mammalian cells for the expression of recombinant human antibodies which exhibit the antigen specificity of the non-human antibody and will undergo posttranslational modifications that will enhance their expression, stability, solubility, or combinations thereof.

Hypervariable regions may be associated with any kind of framework regions, e.g., of human origin. Suitable framework regions were described in Kabat et al. The CDRs may be added to a human antibody framework, such as those described in U.S. Pat. No. 7,456,260, issued to Rybak, et al. which teaches new human variable chain framework regions and humanized antibodies comprising the framework regions, relevant portions and framework sequences incorporated herein by reference. To accomplish the engraftment at a genetic level, the present invention also includes the underlying nucleic acid sequences for the VL and VH regions as well as the complete antibodies and the humanized versions thereof.

Monoclonal antibodies raised against the human IL-18R protein are typically developed in a non-human system e.g., in mice, and as such are typically non-human proteins. As a direct consequence of this, a xenogenic antibody as produced by a hybridoma, when administered to humans, elicits an undesirable immune response that is predominantly mediated by the constant part of the xenogenic immunoglobulin. Xenogeneic antibodies tend to elicit a host immune response, thereby limiting the use of such antibodies, as they cannot be administered over a prolonged period of time. Therefore, it is particularly useful to use single chain, single domain, chimeric, CDR-grafted, or especially human antibodies that are not likely to elicit a substantial allogenic response when administered to humans. The present invention includes antibodies with minor changes in an amino acid sequence such as deletion, addition or substitution of one, a few or even several amino acids which are merely allelic forms of the original protein having substantially identical properties. The constant part of a human heavy chain may be of the, e.g., α1, α2, γ1, γ2, γ3, γ4, μ, β2, or δ or ε type, preferably of the γ-type, whereas the constant part of a human light chain may be of the κ or λ type (which includes the λ1, λ2 and λ3 subtypes) but is preferably of the κ type. The amino acid sequences of the general locations of the variable and constant domains are well known in the art and generally follow the Kabat nomenclature.

The inhibition of the binding of IL-18 to its receptor (IL-18R) may be conveniently tested in various assays including such assays are described hereinafter in the text. By the term “to the same extent” is meant that the reference and the equivalent molecules exhibit, on a statistical basis, essentially identical IL-18R binding inhibition curves in one of the assays referred to above. For example, the assay used may be an assay of competitive inhibition of binding of IL-18R by the binding molecules of the invention.

An IL-18R binding molecule of the invention may be produced by recombinant DNA techniques. In view of this, one or more DNA molecules encoding the binding molecule must be constructed, placed under appropriate control sequences and transferred into a suitable host organism for expression.

The present state of the art is such that the skilled worker in the art can synthesize the DNA molecules of the invention given the information provided herein, i.e., the amino acid sequences of the hypervariable regions and the DNA sequences coding for them. A method for constructing a variable domain gene is for example described in EPA 239 400, relevant portions incorporated herein by reference. Briefly, a gene encoding a variable domain of a MAb is cloned. The DNA segments encoding the framework and hypervariable regions are determined and the DNA segments encoding the hypervariable regions are removed so that the DNA segments encoding the framework regions are fused together with suitable restriction sites at the junctions. The restriction sites may be generated at the appropriate positions by mutagenesis of the DNA molecule by standard procedures. Double stranded synthetic CDR cassettes are prepared by DNA synthesis according to the sequences given in SEQ ID NOS: 1 and 3 or 2 and 4 (amino acid and nucleic acid sequences, respectively). These cassettes are often provided with sticky ends so that they can be ligated at the junctions of the framework.

It is not necessary to have access to the mRNA from a producing hybridoma cell line in order to obtain a DNA construct coding for the IL-18R binding molecules of the invention. For example, PCT Application No. WO 90/07861 gives full instructions for the production of an antibody by recombinant DNA techniques given only written information as to the nucleotide sequence of the gene, relevant portions incorporated herein by reference. Briefly, the method comprises the synthesis of a number of oligonucleotides, their amplification by the PCR method, and their splicing to give the desired DNA sequence.

As used herein, the term “vector” is used in two different contexts. When using the term “vector” with reference to a vaccine, a vector is used to describe a non-antigenic portion that is used to direct or deliver the antigenic portion of the vaccine. For example, an antibody or fragments thereof may be bound to or form a fusion protein with the antigen that elicits the immune response. For cellular vaccines, the vector for delivery and/or presentation of the antigen is the antigen presenting cell, which is delivered by the cell that is loaded with antigen. In certain cases, the cellular vector itself may also process and present the antigen(s) to T cells and activate an antigen-specific immune response. When used in the context of nucleic acids, a “vector” refers a construct, which is capable of delivering, and preferably expressing, one or more genes or polynucleotide sequences of interest in a host cell. Examples of vectors include, but are not limited to, viral vectors, naked DNA or RNA expression vectors, DNA or RNA expression vectors associated with cationic condensing agents, DNA or RNA expression vectors encapsulated in liposomes, and certain eukaryotic cells, such as producer cells.

As used herein, the terms “stable” and “unstable” when referring to proteins is used to describe a peptide or protein that maintains its three-dimensional structure and/or activity (stable) or that loses immediately or over time its three-dimensional structure and/or activity (unstable). As used herein, the term “insoluble” refers to those proteins that when produced in a cell (e.g., a recombinant protein expressed in a eukaryotic or prokaryotic cell or in vitro) are not soluble in solution absent the use of denaturing conditions or agents (e.g., heat or chemical denaturants, respectively). The antibody or fragment thereof and the linkers taught herein have been found to convert antibody fusion proteins with the peptides from insoluble and/or unstable into proteins that are stable and/or soluble. Another example of stability versus instability is when the domain of the protein with a stable conformation has a higher melting temperature (Tm) than the unstable domain of the protein when measured in the same solution. A domain is stable compared to another domain when the difference in the Tm is at least about 2° C., more preferably about 4° C., still more preferably about 7° C., yet more preferably about 10° C., even more preferably about 15° C., still more preferably about 20° C., even still more preferably about 25° C., and most preferably about 30° C., when measured in the same solution.

As used herein, “polynucleotide” or “nucleic acid” refers to a strand of deoxyribonucleotides or ribonucleotides in either a single- or a double-stranded form (including known analogs of natural nucleotides). A double-stranded nucleic acid sequence will include the complementary sequence. The polynucleotide sequence may encode variable and/or constant region domains of immunoglobulin that are formed into a fusion protein with one or more linkers. For use with the present invention, multiple cloning sites (MCS) may be engineered into the locations at the carboxy-terminal end of the heavy and/or light chains of the antibodies to allow for in-frame insertion of peptide for expression between the linkers. As used herein, the term “isolated polynucleotide” refers to a polynucleotide of genomic, cDNA, or synthetic origin or some combination thereof. By virtue of its origin the “isolated polynucleotide” (1) is not associated with all or a portion of a polynucleotide in which the “isolated polynucleotides” are found in nature, (2) is operably linked to a polynucleotide which it is not linked to in nature, or (3) does not occur in nature as part of a larger sequence. The skilled artisan will recognize that to design and implement a vector can be manipulated at the nucleic acid level by using techniques known in the art, such as those taught in Current Protocols in Molecular Biology, 2007 by John Wiley and Sons, relevant portions incorporated herein by reference. Briefly, the encoding nucleic acid sequences can be inserted using polymerase chain reaction, enzymatic insertion of oligonucleotides or polymerase chain reaction fragments in a vector, which may be an expression vector. To facilitate the insertion of inserts at the carboxy terminus of the antibody light chain, the heavy chain, or both, a multiple cloning site (MCS) may be engineered in sequence with the antibody sequences.

As used herein, the term “polypeptide” refers to a polymer of amino acids and does not refer to a specific length of the product; thus, peptides, oligopeptides, and proteins are included within the definition of polypeptide. This term also does not refer to or exclude post expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. The term “domain,” or “polypeptide domain” refers to that sequence of a polypeptide that folds into a single globular region in its native conformation, and that may exhibit discrete binding or functional properties.

The term “fusion protein” refers to the expression product of two or more nucleic acid molecules that are not natively expressed together as one expression product. Fusion proteins can be made at the nucleic acid coding level by placing, in-line and in the correct coding frame, the two or more sequences of the portions of the proteins or peptides. Fusion proteins are synthesized by methods known to those of skill in the art including, e.g., solid phase protein synthesis, and by molecular techniques that permit the manipulation of DNA in vitro, including polymerase chain reaction (PCR) and oligonucleotide-directed mutagenesis. A fusion protein for use with the present invention is an immunotoxin, which includes an antigen binding portion, in this case an IL-18R binding protein, and a toxin.

A polypeptide or amino acid sequence “derived from” a designated nucleic acid sequence refers to a polypeptide having an amino acid sequence identical to that of a polypeptide encoded in the sequence, or a portion thereof wherein the portion consists of at least 3-5 amino acids, preferably at least 4-7 amino acids, more preferably at least 8-10 amino acids, and even more preferably at least 11-15 amino acids, or which is immunologically identifiable with a polypeptide encoded in the sequence. This terminology also includes a polypeptide expressed from a designated nucleic acid sequence.

Expression vectors comprising a suitable promoter or genes encoding heavy and light chain constant parts are publicly available. Thus, once a DNA molecule of the invention is prepared it may be conveniently transferred in an appropriate expression vector. DNA molecules encoding single chain antibodies may also be prepared by standard methods, for example, as described in WO 88/1649. In view of the foregoing, no hybridoma or cell line deposit is necessary to comply with the criteria of sufficiency of description.

For example, first and second DNA constructs are made that bind specifically to IL-18R. Briefly, a first DNA construct encodes a light chain or fragment thereof and comprises, a) a first part which encodes a variable domain comprising alternatively framework and hypervariable regions, the hypervariable regions being in sequence CDR1L, CDR2L and CDR3L; this first part starting with a codon encoding the first amino acid of the variable domain and ending with a codon encoding the last amino acid of the variable domain, and b) a second part encoding a light chain constant part or fragment thereof which starts with a codon encoding the first amino acid of the constant part of the heavy chain and ends with a codon encoding the last amino acid of the constant part or fragment thereof, followed by a stop codon. A second part encodes the constant part of a human heavy chain, more preferably the constant part of the human γ1 chain. This second part may be a DNA fragment of genomic origin (comprising introns) or a cDNA fragment (without introns).

The second DNA construct encodes a heavy chain or fragment thereof and comprises, a) a first part which encodes a variable domain comprising alternatively framework and hypervariable regions; the hypervariable regions being CDR1H and optionally CDR2H and CDR3H; this first part starting with a codon encoding the first amino acid of the variable domain and ending with a codon encoding the last amino acid of the variable domain, and b) a second part encoding a heavy chain constant part or fragment thereof which starts with a codon encoding the first amino acid of the constant part of the light chain and ends with a codon encoding the last amino acid of the constant part or fragment thereof followed by a stop codon.

The invention also includes IL-18R binding molecules in which one or more of the residues of CDR1L, CDR2L, CDR3L, CDR1H, CDR2H or CDR3H or the frameworks, typically only a few (e.g. FR1-4L or H), are changed from the residues by, e.g., site directed mutagenesis of the corresponding DNA sequences. The invention includes the DNA sequences coding for such changed IL-18R binding molecules.

Each of the DNA constructs are placed under the control of suitable control sequences, in particular under the control of a suitable promoter. Any kind of promoter may be used, provided that it is adapted to the host organism in which the DNA constructs will be transferred for expression. However, if expression is to take place in a mammalian cell, an immunoglobulin gene promoter may be used in B cells. The first and second parts may be separated by an intron, and, an enhancer may be conveniently located in the intron between the first and second parts. The presence of such an enhancer that is transcribed but not translated, may assist in efficient transcription. In particular embodiments of the present invention the first and second DNA constructs comprise the enhancer of, e.g., a heavy chain human gene.

The desired antibody may be produced in a cell culture or in a transgenic animal. A suitable transgenic animal may be obtained according to standard methods that include micro injecting into eggs the first and second DNA constructs placed under suitable control sequences transferring the so prepared eggs into appropriate pseudo-pregnant females and selecting a descendant expressing the desired antibody.

The invention also provides an expression vector able to replicate in a prokaryotic or eukaryotic cell line, which comprises at least one of the DNA constructs above described. Each expression vector containing a DNA construct is then transferred into a suitable host organism. When the DNA constructs are separately inserted on two expression vectors, they may be transferred separately, i.e., one type of vector per cell, or co-transferred, this latter possibility being preferred. A suitable host organism may be a bacterium, a yeast or a mammalian cell line, this latter being preferred. More preferably, the mammalian cell line is of lymphoid origin, e.g., a myeloma, hybridoma or a normal immortalized B-cell, which conveniently does not express any endogenous antibody heavy or light chain.

When the antibody chains are produced in a cell culture, the DNA constructs must first be inserted into either a single expression vector or into two separate but compatible expression vectors, the latter possibility being preferred. For expression in mammalian cells it is preferred that the coding sequence of the IL-18R binding molecule is integrated into the host cell DNA within a locus, which permits or favors high level expression of the IL-18R binding molecule.

In a further aspect of the invention there is provided a process for the product of a IL-18R binding molecule that comprises: (i) culturing an organism which is transformed with an expression vector as defined above; and (ii) recovering the IL-18R binding molecule from the culture.

To use the anti-IL-18R antibody of the present invention for treatment indications, the appropriate dosage will, of course, vary depending upon, for example, the antibody disclosed herein to be employed, the host, the mode of administration and the nature and severity of the condition being treated. However, in prophylactic use, satisfactory results are generally found at dosages from about 0.05 mg to about 10 mg per kilogram body weight more usually from about 0.1 mg to about 5 mg per kilogram body weight. The frequency of dosing for prophylactic uses will normally be in the range from about once per week up to about once every 3 months, more usually in the range from about once every 2 weeks up to about once every 10 weeks, e.g., once every 4 to 8 weeks. The anti-IL-18R antibody of the present invention can be administered parenterally, intravenously, e.g., into the antecubital or other peripheral vein, intramuscularly, or subcutaneously.

As used herein, “pharmaceutically acceptable carrier” refers to any material that when combined with an immunoglobulin (Ig) fusion protein of the present invention allows the Ig to retain biological activity and is generally non-reactive with the subject's immune system. Examples include, but are not limited to, standard pharmaceutical carriers such as a phosphate buffered saline solution, water, emulsions such as an oil/water emulsion, and various types of wetting agents. Certain diluents may be used with the present invention, e.g., for aerosol or parenteral administration, that may be phosphate buffered saline or normal (0.85%) saline.

Pharmaceutical compositions of the invention may be manufactured in conventional manner, e.g., in a lyophilized form. For immediate administration it is dissolved in a suitable aqueous carrier, for example sterile water for injection or sterile buffered physiological saline. If it is considered desirable to make up a solution of larger volume for administration by infusion rather as a bolus injection, it is advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution.

One embodiment of the present invention provides an immunoconjugate comprising a humanized antibody of the invention, e.g., a humanized anti-IL-18R antibody or fragments thereof, linked to one or more effector molecules, antigen(s) and/or a detectable label(s). Examples of fragments of antibodies include e.g., Fv, scFv, Fab′ and F(ab′)2. Effector molecule is a therapeutic molecule such as, for example, one or more peptides that comprise one or more toxins, antibiotics, radioactive isotopes and nucleolytic enzymes.

Exemplary toxins include, but are not limited to, ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin. Examples of small molecules include, but are not limited to, chemotherapeutic compounds such as auristatin; maytansinoid; calicheamicin; MMAE (mono-methyl auristatin E); MMAF and AEVB (auristatin E valeryl benzylhydrazone); and AFP (Auristatin F phenylene diamine. Exemplary cytotoxic agent is from the group consisting of ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin. Exemplary radioisotopes include, but are not limited to, 64Cu; 67Cu; 90Y; 1231; 1311; 186Re; 188Re; 212Pb; 212Bi; 211At; and 213Bi. Yet other examples of cytotoxic agent include nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

IL-18 receptor as a novel target of regulatory T cells in cancer. The present inventors have previously demonstrated that tumor antigen-specific regulatory T cells (T regs) are present in blood of metastatic melanoma patients¹. Such specific regulatory T cells secrete IL-10 and express FoxP3, a master transcription factor for Tregs, and display suppressive activity toward effector T cells, such as cytotoxic T cells.

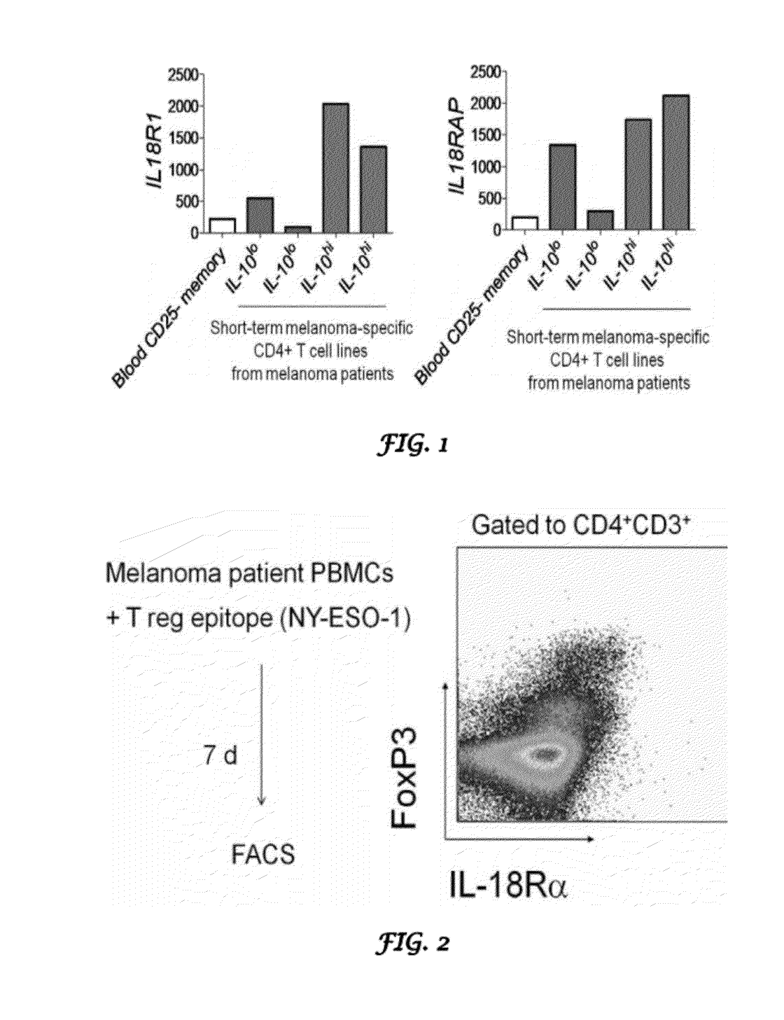

The present inventors have generated short-term melanoma-specific T reg cell lines that secrete IL-10, and non-T reg cell lines that do not secrete IL-10, from PBMCs of melanoma patients. To analyze the genes dominantly expressed by IL-10+ T regs, we performed microarray analysis. Blood memory CD25− (non-T reg) CD4+ T cells were used as a control. Briefly, melanoma-specific T regs express IL-18 receptors, the present inventors cultured PBMCs (5×10⁵ cells/well) from melanoma patients (FIG. 1 shows results with Pt#006-025-002) for 7 days with T reg epitopes (15-mer, 25 microM/ml). FIG. 1 shows the result with NY-ESO-1 peptide#19), which were identified with EpiMax assay. EpiMax is a combinatory strategy developed at BIIR to assess the global repertoire of antigen-specific T cells. EpiMax allows identification of 1) the breadth (T cell epitopes) and 2) the type (cytokine production patterns) of antigen-specific CD4+ and CD8+ T cells. Briefly, overlapping peptides covering the sequence of a given antigen are tested for their ability to induce PBMCs to secrete cytokines and cause T cell proliferation. An outline of the procedure is described herein below:

CFSE-labeled PBMCs are cultured with clusters of 15-17mer overlapping peptides.

Cytokines/chemokines (IL-2, IL-5, IL-10, IL-13, IL-17 and IP-10) secreted into the culture supernatant during 48 h are measured with Luminex. T cell type can be determined according to the pattern of cytokine secretion: (1) Type 1 cells (Tc1/Th1) by IP-10 upregulation; (2) Type 2 cells (Th2/Tc2) by IL-13 upregulation; (3) Type 17 cells (Th17/Tc17) by IL-17 upregulation, and (4) Type 10 cells (IL-10-producing Th1 cells, IL-10-producing Th2 cells, Tr1 cells, and T regs) by IL-10 upregulation.

CD4+ and CD8+ T cell proliferation is analyzed at day 8 of PBMC culture based on the CFSE dilution. This step determines the T cell subsets (CD4+ or CD8+) responding to the peptide clusters together with the proliferative capacity of the specific T cells.

To identify a T cell epitope, PBMCs are cultured with single peptides from the positive cluster(s), and cytokine secretion (day 2) and T cell proliferation (day 8) are assessed as described above. The present inventors use EpiMax regularly to determine antigen-specific T cell repertoires in many studies, including cancer, multiple infectious diseases, and autoimmune diseases.

The inventors analyzed the expression of IL-18 receptor alpha chain (IL-18Ra-PE. eBioscience, H44) and FoxP3 (FoxP3-APC. eBioscience, PCH101) in CD4+ T cell population (analyzed with anti-CD3-FITC, UCTH1; anti-CD4-PerCP, SK3).

FIG. 1 shows the expression level of IL-18R1 and IL-18R2 transcript. IL-10+ T reg cell lines expressed much higher levels of IL-18R1 and IL-18R2 than IL-10-non-T reg cell lines or blood memory non T regs. IL-10⁺ melanoma-specific T reg cell lines express higher levels of IL-18R1 transcripts. CFSE-labeled PBMCs (5×10⁵ cells/well/500 microL) of a melanoma patient (Pt#002-094-004) were cultured with Survivin peptide#15 (LAQCFFCFKELEGW) (SEQ ID NO: 1), NY-ESO-1 peptide#23 (EFYLAMPFATPMEAE) (SEQ ID NO.: 2), or TRP-1 peptide#113 (NTEMFVTAPDNLGYT) (SEQ ID NO.: 3) (15mers, 25 microM) for 7 days, and CFSE-CD4+ T cells were sorted into 96-well plates (1-3 cells/well). Sorted cells were cultured for 3 weeks with irradiated ICOS-L-transfected L cells in the presence of anti-CD3 (OKT3) and IL-2 (100 IU/ml). CD4+ T cells secreted >400 pg/ml IL-10 were pooled as IL-10^{hi} CD4+ T cells, while CD4+ T cells secreted <50 pg/ml IL-10 were pooled as IL-10^{lo} CD4+ T cells. Blood CD25-CD45RA− CD4+ T cells were sorted from the same patient PBMCs as a control. The raw intensity of IL-18R1 and IL-18RAP (gene for IL-18R2) in microarray analysis is shown.

Activated blood melanoma-specific T regs express IL-18Rα. PBMCs (5×10⁵ cells/well) from a melanoma patient (Pt#006-025-002) were cultured for 7 days with a T reg epitope (15-mer NY-ESO-1 peptide#19. 25 microM/ml) and the expression of IL-18 receptor alpha chain (IL-18Rα-PE) and FoxP3 (FoxP3-APC) was analyzed in CD4+ T cell population. Gated to CD3+CD4+ T cells. FIG. 2 shows a representative result. It was found that activated FoxP3+ T regs (which express highest levels of FoxP3) expressed the highest levels of IL-18Ra among CD4+ T cells.

To determine whether blood T regs in melanoma patients respond to IL-18, the present inventors sorted memory CD25-(non-Treg) CD4+ T cells and CD25+CD4+ T regs from PBMCs of a melanoma patient (#002-094-002), and stimulated them with anti-CD3/CD28 mAb-coated beads (Human T-Activator CD3/CD28 for cell expansion and activation. Dynal) in the presence or absence of IL-18 (100 ng/ml, R&D). FIG. 3 shows IFN-γ secretion during 48 h stimulation (5×10⁴ cells/well/200 microL). IFN-γ secretion during 48 h stimulation was measured (5×10⁴ cells/well/200 microL). The T-test showed that as expected, T regs secreted much less IFN-γ overall when compared to non-Tregs. However, supplementation of IL-18 resulted in the significant upregulation of IFN-γ secretion by T regs. This observation shows that blood T regs in blood of metastatic melanoma patients express functional IL-18 receptors.

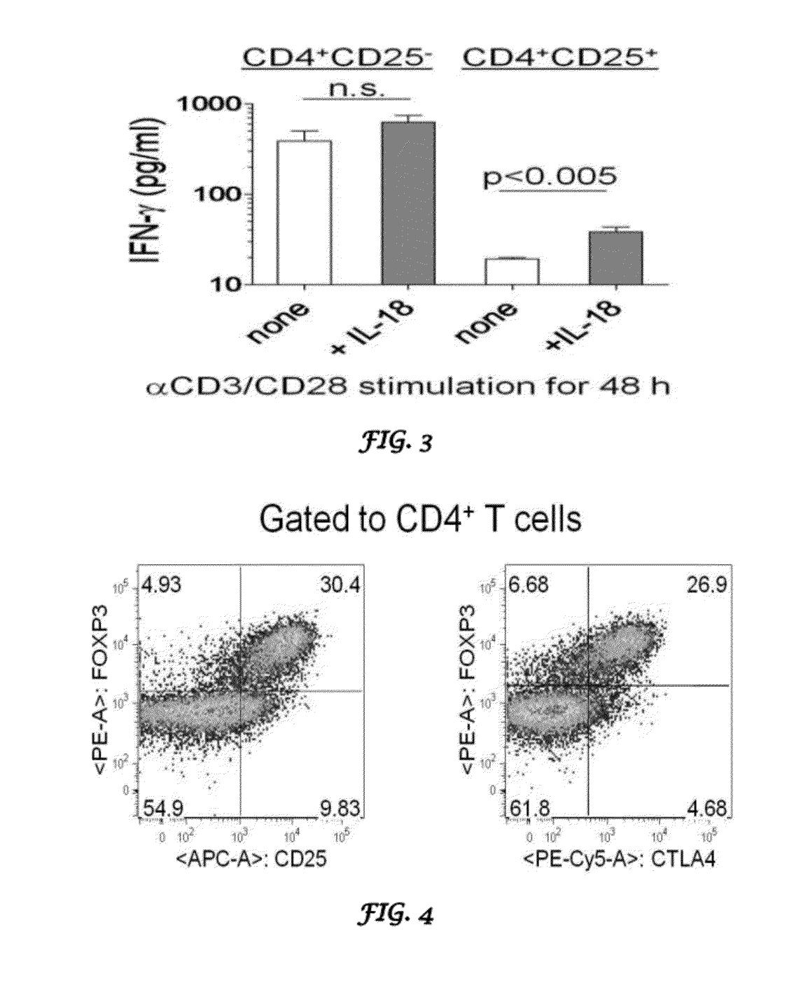

Next, the present inventors analyzed whether T regs found in metastatic melanoma tumors express IL-18Rα. Single cell suspension was obtained from a fresh metastatic melanoma tumor resected from an axillary lymph node of a patient (009-075-001-016), stained (1×10⁶ cells) with anti-FoxP3-PE (eBioscience, PCH101), anti-CTLA4-PE Cy5 (BD Pharmingen, BNI3), anti-CD25-APC (BD Pharmingen, M-A251), anti-CD3-AF700 (BD Pharmingen, UCTH1), anti-CD4-PB (eBioscience, OKT4), and anti-CD45-PO (Invitrogen, HI30), and analyzed with FACSCantoII. A large fraction of CD4+ T cells (gated to CD45+CD3+CD4+ cells) found in tumors expressed FoxP3, CD25, and CTLA-4, thus represents T regs (FIG. 4).

FIG. 4 shows that T regs are found in metastatic melanoma tumors. Single cell suspension was obtained from a fresh metastatic melanoma tumor, stained (1×10⁶ cells) with anti-FoxP3-PE, anti-CTLA4-PE Cy5, anti-CD25-APC, anti-CD3-AF700, anti-CD4-PB, and anti-CD45-PO and analyzed with FACSCantoII. Gated to CD45+CD3+CD4+ T cells.

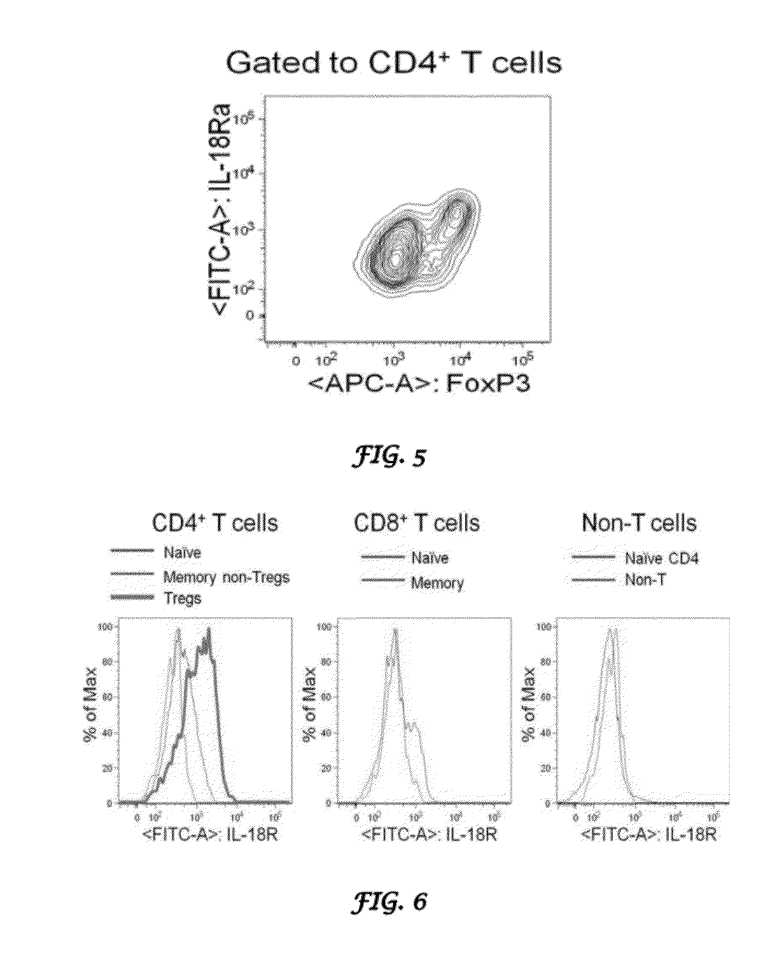

It was also found that T regs found in metastatic melanoma tumors express high levels of IL-18Rα (IL-18Rα-PE. eBioscience, H44) (FIG. 5). FIG. 5 shows that T regs found in metastatic melanoma tumors express high levels of IL-18Rα. Single cell suspension was obtained from a fresh metastatic melanoma tumor, stained (1×10⁶ cells) with anti-FoxP3-APC, anti-CD3-AF700, anti-CD4-PB, anti-IL-18Rα-PE, and anti-CD45-PO (Invitrogen, HI30), and analyzed with FACSCantoII. Gated to CD45+CD3+CD4+ T cells.

FIG. 6 left panel shows a flow cytometry analysis of IL-18Rα expression on FoxP3+ T regs, FoxP3-CD45RA-memory CD4+ T cells, and CD45RS+naïve CD4+ T cells that were present in the metastatic melanoma tumor. Single cell suspension of tumor cells (1×10⁶ cells) were stained with anti-18Rα-FITC (eBioscience, H44), anti-FoxP3-APC (eBioscience, PCH101), anti-CTLA4-PE Cy5 (BD Pharmingen, BNI3), anti-CD25-APC (BD Pharmingen, M-A251), anti-CD3-APC AF750 (BD Pharmingen, UCTH1), LIVE/DEAD® Fixable Violet Dead Cell Stain Kit (Invitrogen) and anti-CD45-PO (Invitrogen, HI30), and analyzed with FACSCantoII. The levels of IL-18Rα expressed by FoxP3+ T regs were the highest among CD4+ T cells (gated to Violet-CD45+CD3+CD4+ cells). It was found that IL-18Rα is expressed by T regs at highest levels in metastatic melanoma tumors. IL-18Rα expression level was analyzed on CD4+ T cell subsets (left), CD8+ T cell subsets (middle), and non-T cell lymphocyte populations (right) infiltrating into a metastatic melanoma tumor.

Furthermore, expression levels of IL-18Rα by FoxP3+ T regs were even higher than memory CD8+ T cells (CD45RA-CD3+CD4− T cells), or other lymphocytes (CD45+CD3− cells, possibly containing NK cells) infiltrating into the same tumor. Thus, IL-18Rα is expressed by T regs at highest levels in metastatic melanoma tumors.

Recent mouse studies demonstrated the presence of FoxP3+ T regs co-expressing T-bet², a master transcription factor of Th1 cells. Such T regs express the chemokine receptor CXCR3, with which they migrate into Th1-inflamatory sites, and suppress inflammatory responses. IFN-g induces mouse Tregs to express T-bet. The present inventors recognized that if IL-18 is a potent co-inducer of IFN-γ, it might be possible that IL-18Rα+FoxP3+ T regs express CXCR3 and T-bet.

The inventors then analyzed the expression of CXCR3 and T-bet in FoxP3+ T regs infiltrating into metastatic melanoma tumors. Single cell suspension of tumor cells (1×10⁶ cells) were stained with anti-18Rα-FITC (eBioscience, H44), anti-CXCR3-PE Cy5 (BD Pharmingen, 106), anti-FoxP3-APC (eBioscience, PCH101), anti-CTLA4-PE Cy5 (BD Pharmingen, BNI3), anti-CD25-APC (BD Pharmingen, M-A251), anti-CD3-APC AF750 (BD Pharmingen, UCTH1), LIVF/DEAD® Fixable Violet Dead Cell Stain Kit (Invitrogen) and anti-CD45-PO (Invitrogen, HI30), and analyzed with FACSCantoII.

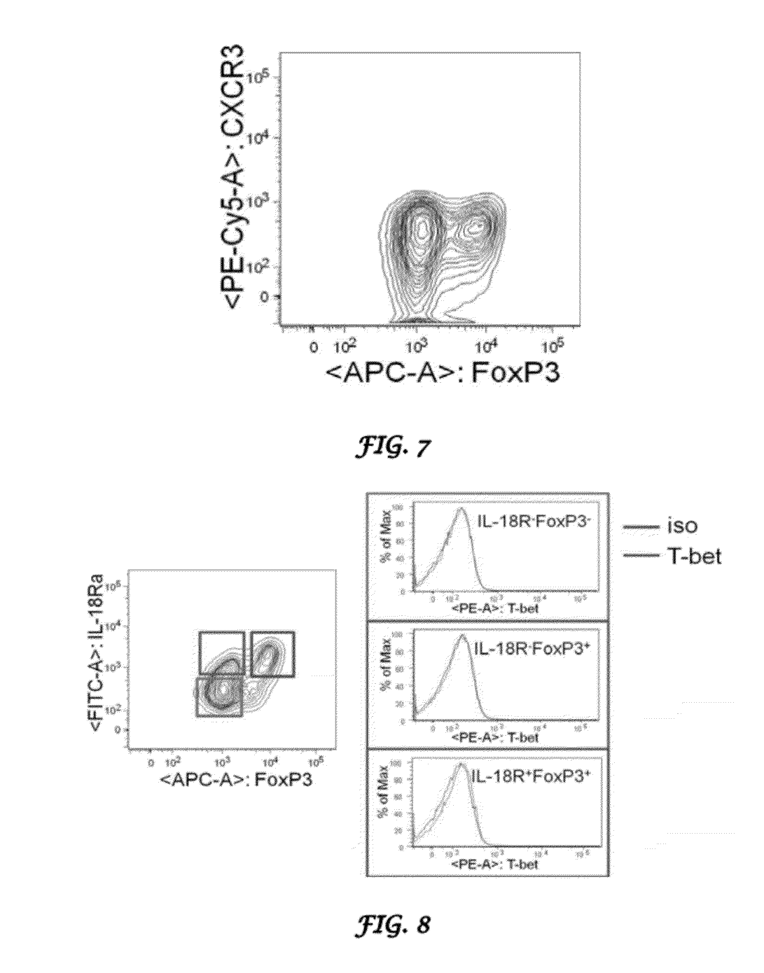

FIG. 7 shows that FoxP3+ T regs indeed co-express high levels of CXCR3. That FoxP3+ T regs was found in metastatic melanoma tumors express CXCR3 as demonstrated by using single cell suspension of tumor cells were stained with anti-18Rα-FITC, anti-CXCR3-PE, anti-FoxP3-APC, anti-CTLA4-PE Cy5, anti-CD25-APC, anti-CD3-APC AF750, LIVF/DEAD® Fixable Violet Dead Cell Stain Kit and anti-CD45-PO, and analyzed with FACSCantoII. Gated to Violet-CD45+CD3+CD4+ T cells.

Furthermore, FoxP3+ T regs expressed T-bet. FIG. 8 shows the expression of T-bet by FoxP3-IL-18Ra−, FoxP3-IL-18Ra+, and FoxP3+IL-18Ra+CD4+ T cells. Only FoxP3+IL-18Ra+CD4+ T cells showed the expression of T-bet. Briefly, single cell suspension of tumor cells (1×10⁶ cells) were stained with anti-18Ra-FITC (eBioscience, H44), anti-T-bet-PE (Santa Cruz, 4B10) or control IgG1-PE (Santa Cruz), anti-FoxP3-APC (eBioscience, PCH101), anti-CTLA4-PE Cy5 (BD Pharmingen, BNI3), anti-CD25-APC (BD Pharmingen, M-A251), anti-CD3-APC AF750 (BD Pharmingen, UCTH1), LIVF/DEAD® Fixable Violet Dead Cell Stain Kit (Invitrogen) and anti-CD45-PO (Invitrogen, HI30), and analyzed with FACSCantoII. The expression of T-bet by FoxP3-IL-18Ra−, FoxP3-IL-18Ra+, and FoxP3+IL-18Ra+CD4+ T cells. Only FoxP3+IL-18Ra+CD4+ T cells showed the expression of T-bet. Gated to CD4+CD3+CD45+ T cells.

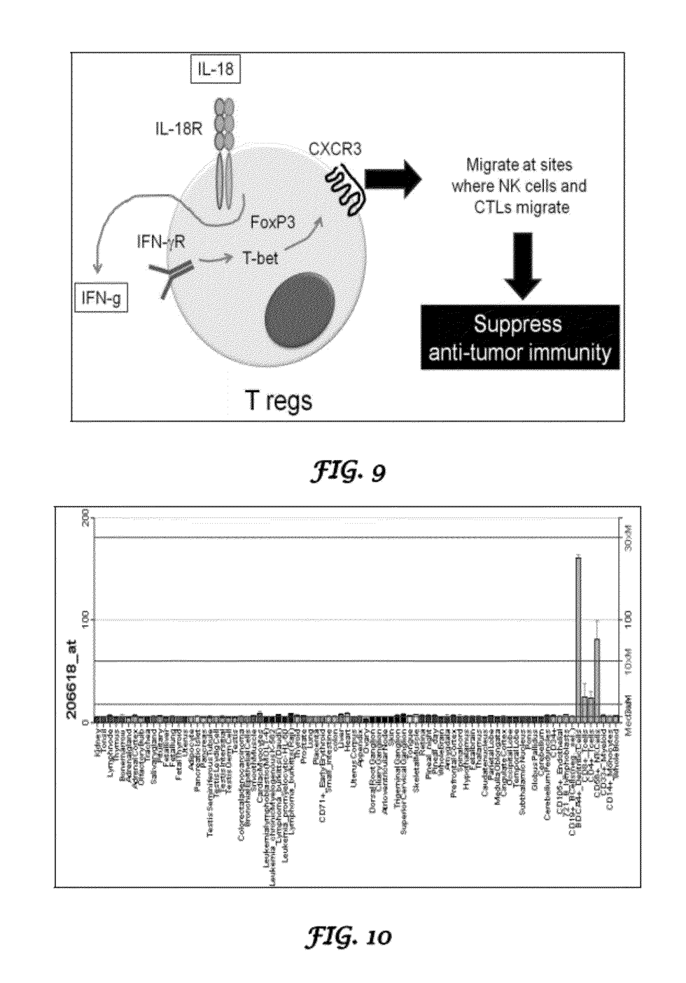

Collectively, these studies demonstrate that: (1) blood tumor antigen-specific T regs in metastatic melanoma patients express functional IL-18 receptor and secrete IFN-γ in response to IL-18 stimulation; (2) T regs express the highest levels of IL-18Ra among tumor-infiltrating lymphocytes; and (3) IL-18Ra-expressing FoxP3+ T regs co-express CXCR3 and T-bet. As IFN-γ induces CD4+ T cells express T-bet and CXCR3, IL-18 might be involved in the generation of CXCR3+ T-bet+ T regs in cancer through the secretion of IFN-γ. Given that NK cells and activated CD8+ T cells express CXCR3, CXCR3+ T regs likely accumulate at the same sites where NK cells and CD8+ T cells migrate, and thus suppress anti-tumor immunity at tumor sites mediated by these effectors (FIG. 9). Thus, IL-18Ra is a target molecule that can be targeted to eliminate tumor-infiltrating T regs, and/or to suppress their functions (migration to tumor sites, suppressive activity, etc.), thereby allowing the immune response against the tumor to proceed without being blocked by the T regs.

IL-18R1 is dominantly expressed by plasmacytoid DCs (pDCs), NK cells, and T cells in humans (FIG. 10). Recent studies show that pDCs infiltrate many human tumors, including melanoma, head and neck cancer, metastatic ovarian carcinoma, non-small cell lung carcinomas (NSCLC), and breast cancer. There, the capacity of type 1 IFN production by pDCs was found to be inhibited by tumors. It was also shown that pDCs do not induce effector T cell responses, but rather promote the differentiation of IL10+CD8+ Tregs in ovarian carcinoma. Thus, treatment targeting IL-18R1 in cancer might also benefit from the elimination and/or suppression of the function of such tolerogeneic pDCs.

It is expected that these observations with metastatic melanoma can be expanded to similar T regs in cancers known to secrete IL-18, or IL-18-rich environment³, including: melanoma, gastric cancer^{4,5}; esophageal cancer; pancreatic cancer; colon cancer; hepatocellular carcinoma; head and neck squamous cell carcinoma (hnscc); lung cancer; breast cancer; ovarian cancer; bladder cancer; renal cell carcinoma; leukemia and other malignant hematological diseases; prostate cancer; and skin tumors including squamous cell carcinoma (SCC).⁶

T regs associated with chronic infectious diseases known to induce IL-18-rich environment might also be a target in this approach, which includes, viruses such as HCV.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,651,855: Regulatory T Cells and Their Use in Immunotherapy and Suppression of Autoimmune Responses.
U.S. Pat. No. 7,115,259: Use of Cytokines and Mitogens to Inhibit Pathological Immune Responses.
U.S. Patent Application Publication No. 20100092488: Method for Treatment or Prevention of Disease Associated with Functional Disorder of Regulatory T Cell.
U.S. Patent Application Publication No. 20100003287: Compositions and Methods Relating to Treatment of Cancer and Infectious Diseases.
1. Vence, L., et al., Circulating tumor antigen-specific regulatory T cells in patients with metastatic melanoma. Proceedings of the National Academy of Sciences of the United States of America 104, 20884-20889 (2007).
2. Koch, M. A., et al., The transcription factor T-bet controls regulatory T cell homeostasis and function during type 1 inflammation. Nature immunology 10, 595-602 (2009).
3. Vidal-Vanaclocha, F. et al., Clinical and experimental approaches to the pathophysiology of interleukin-18 in cancer progression. Cancer Metastasis Rev 25, 417-434, doi:10.1007/s10555-006-9013-3 (2006).
4. Kang, J. S., et al., Interleukin-18 increases metastasis and immune escape of stomach cancer via the downregulation of CD70 and maintenance of CD44. Carcinogenesis 30, 1987-1996, doi:bgp158 [pii] 10.1093/carcin/bgp158 (2009).
5. Kim, K. E., et al., Interleukin-18 is a critical factor for vascular endothelial growth factor-enhanced migration in human gastric cancer cell lines. Oncogene 26, 1468-1476, doi:1209926 [pii] 10.1038/sj.one.1209926 (2007).
6. Park, H, et al., Enhanced IL-18 expression in common skin tumors. Immunology letters 79, 215-219, doi:S0165247801002784 [pii] (2001).
7. Colombo, M. P. and Piconese, S., Regulatory T-cell inhibition versus depletion: the right choice in cancer immunotherapy. Nature Reviews Cancer 7, 880-887 (2007).
8. Beyer, M. and Schultze, J. M., Regulatory T cells in cancer. Blood 108(3), 804-811 (2006).

What is claimed is:

1. A method of preventing, inhibiting or reducing tumor cell growth comprising administering an effective amount of an active agent comprising an IL-18 Receptor (IL-18R) binding molecule to a cell or an animal in need thereof sufficient to kill one or more tumor specific regulatory T cells.

2. The method of claim 1, further comprising the step of injecting the patient with a tumor cell-specific dendritic cell vaccine.

3. The method of claim 1, wherein the active agent comprises an anti-IL-18R antibody or fragments thereof, an IL-18R antagonist, an IL-18 Fc or an IL-18 Toxin.

4. The method of claim 3, wherein the antibody comprises an Fab, Fv, scFv, Fab′, and F(ab′)₂ antibody fragment.

5. The method of claim 3, wherein the antibody is a humanized antibody.

6. The method of claim 3, wherein the antibody is an immunotoxin that comprises an IL-18 Receptor antigen binding fragment and a cytotoxic agent selected from the group consisting of toxins, antibiotics, radioactive isotopes, and nucleolytic enzymes.

7. The method of claim 6, wherein the cytotoxic agent is a toxin selected from the group consisting of auristatin; maytansinoid; calicheamicin; mono-methyl auristatin E; mono-methyl auristatin F; auristatin E valeryl benzylhydrazone; and Auristatin F phenylene diamine.

8. The method of claim 6, wherein the cytotoxic agent is from the group consisting of ricin; abrin; alpha toxin; saporin; ribonuclease (RNase); deoxyribonuclease; Staphylococcal enterotoxin-A; pokeweed antiviral protein; gelonin; diphtheria toxin; Pseudomonas exotoxin; and Pseudomonas endotoxin.

9. The method of claim 6, wherein the cytotoxic agent is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi.

10. The method of claim 6, wherein the cytotoxic agent is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

11. The method of claim 1, wherein the active agent further comprises an antitumor drug.

12. The method of claim 1, wherein the T cells are IL-18R^{high}, FoxP3+, CXCR3+.

13. The method of claim 1, wherein the T cells express at least one of CTLA-4, CD25, CD28, and T-bet.

14. The method of claim 1, wherein the tumor is at least one of a melanoma cancer; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors; and a squamous cell carcinoma.

15. A pharmaceutical composition for use in preventing, inhibiting or reducing tumor cell growth comprising an effective amount of an active agent that kills one or more IL-18 Receptor expressing T cells in admixture with a suitable diluent or carrier.

16. The composition of claim 15, wherein the composition further comprises the step of injecting the patient with a tumor cell-specific dendritic cell vaccine.

17. The composition of claim 15, wherein the active agent comprises an anti-IL-18R antibody or fragments thereof, an IL-18R antagonist, an IL-18 Fc or an IL-18 Toxin.

18. The composition of claim 17, wherein the antibody comprises an Fab, Fv, scFv, Fab′, and F(ab′)₂ antibody fragment.

19. The composition of claim 17, wherein the antibody is a humanized antibody.

20. The composition of claim 17, wherein the antibody is an immunotoxin that comprises an IL-18 Receptor antigen binding fragment and a cytotoxic agent selected from the group consisting of toxins, antibiotics, radioactive isotopes, and nucleolytic enzymes.

21. The composition of claim 20, wherein the cytotoxic agent is a toxin selected from the group consisting of auristatin; maytansinoid; calicheamicin; mono-methyl auristatin E; mono-methyl auristatin F; auristatin E valeryl benzylhydrazone; and Auristatin F phenylene diamine.

22. The composition of claim 20, wherein the cytotoxic agent is from the group consisting of ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin.

23. The composition of claim 20, wherein the cytotoxic agent is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi.

24. The composition of claim 20, wherein the cytotoxic agent is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

25. The composition of claim 15, wherein the T cells are Regulator T cells that are FoxP3+, CXCR3+.

26. The composition of claim 15, wherein the active agent further comprises an antitumor drug.

27. The composition of claim 15, wherein the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer;bladder cancer; renal cell carcinoma; leukemia; malignant hematological diseases; prostate cancer; skin tumors; and a squamous cell carcinoma.

28. A method of reducing tumor cell growth or metastasis comprising administering an active agent that kills IL-18 R^{high} Regulatory T cells.

29. The method of claim 28, wherein the T cells are IL-18R^{high}, FoxP3+, CXCR3+.

30. The method of claim 28, wherein the T cells express at least one of CTLA-4, CD25, CD28, and T-bet.

31. A method comprising: screening cancer patients suspected of having a tumor to identify those in which expression of IL-18R is upregulated on T cells and administering an active agent that kills the T cells.

32. The method of claim 31, further comprising the step of injecting the patient with a tumor cell-specific dendritic cell vaccine.

33. The method of claim 31, wherein the active agent comprises an anti-IL-18R antibody or fragments thereof, an IL-18R antagonist, an IL-18 Fc, or an IL-18 Toxin.

34. The method of claim 33, wherein the antibody comprises an immunotoxin.

35. The method of claim 33, wherein the antibody comprises an Fab, Fv, scFv, Fab′, and F(ab′)₂ antibody fragment.

36. The method of claim 33, wherein the antibody is a humanized antibody.

37. The method of claim 33, wherein the immunotoxin comprises an IL-18 Receptor antigen binding fragment and a cytotoxic agent selected from the group consisting of toxins, antibiotics, radioactive isotopes, and nucleolytic enzymes.

38. The method of claim 37, wherein the cytotoxic agent is a toxin selected from the group consisting of auristatin; maytansinoid; calicheamicin; mono-methyl auristatin E; mono-methyl auristatin F; auristatin E valeryl benzylhydrazone; and Auristatin F phenylene diamine.

39. The method of claim 31, wherein the active agent is from the group consisting of ricin; abrin; alpha toxin; saporin; ribonuclease (RNase); DNase I; Staphylococcal enterotoxin-A; pokeweed antiviral protein; gelonin; diphtheria toxin; Pseudomonas exotoxin; and Pseudomonas endotoxin.

40. The method of claim 31, wherein the active agent is a radionuclide selected from the group consisting of ⁶⁴Cu; ⁶⁷Cu; ⁹⁰Y; ¹²³I; ¹³¹I; ¹⁸⁶Re; ¹⁸⁸Re; ²¹²Pb; ²¹²Bi; ²¹¹At; and ²¹³Bi.

41. The method of claim 31, wherein the active agent is selected from the group consisting of nitrogen mustards; ethylenimine derivatives; alkyl sulfonates; nitrosoureas; triazenes; folic acid analogs; anthracyclines; taxanes; COX-2 inhibitors; pyrimidine analogs; purine analogs; antimetabolites; antibiotics; epipodophyllotoxins; platinum coordination complexes; vinca alkaloids; substituted ureas; methyl hydrazine derivatives; endostatin; taxol; camptothecin; oxaliplatin; doxorubicin; and doxorubicin analogs.

42. The method of claim 31, wherein the active agent further comprises an antitumor drug.

43. The method of claim 31, wherein the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors, and a squamous cell carcinoma.

44. A method for determining whether a tumor will respond to anti-IL-18 Receptor therapy comprising:
isolating T cells from a subject suspected of having a tumor; and
determining a level of expression of IL-18R on the T cells isolated from the subject, wherein an increase in the level of IL-18R on the T cells as compared to a normal subject is indicative that the patient will respond to anti-Regulatory T cell therapy.

45. The method of claim 44, further comprising the step of injecting the patient with a tumor cell-specific dendritic cell vaccine.

46. The method of claim 44, wherein the T cells are also evaluated for the expression of at least one of CTLA-4, CD25, CD28, FoxP3, CXCR3, and T-bet.

47. The method of claim 44, further comprising the step of treating the subject with an anti-IL-18 Receptor antibody or fragments thereof, an IL-18R antagonist, an IL-18 Fc or an IL-18 Toxin.

48. The method of claim 44, further comprising the step of treating the patient with an antitumor drug.

49. The method of claim 44, wherein the tumor is at least one of a melanoma; gastric cancer; esophageal cancer; pancreatic cancer; colon cancer; hepatocellular carcinoma; head and neck squamous cell carcinoma; lung cancer; breast cancer; ovarian cancer; bladder cancer; renal cell carcinoma; leukemia; malignant hematological diseases; prostate cancer; skin tumors; and a squamous cell carcinoma.

50. The method of claim 44, wherein the T cells are IL-18R^{high}, FoxP3+, CXCR3+.

51. The method of claim 44, wherein the T cells express at least one of CTLA-4, CD25, CD28, and T-bet.

52. A method of treating melanoma comprising administering an effective amount of an active agent comprises an IL-18 Receptor (IL-18R) binding molecule to a cell or an animal in need thereof sufficient to kill specific regulatory T cells in the melanoma.

53. The method of claim 52, further comprising the step of injecting the patient with a melanoma-specific dendritic cell vaccine.

54. A pharmaceutical composition for use in inhibiting or reducing melanoma cells growth comprising an effective amount of an active agent that kills IL-18 Receptor expressing T cells in the melanoma in an admixture with a suitable diluent or carrier.

55. The composition of claim 54, wherein the composition further comprises the step of injecting the patient with a tumor cell-specific dendritic cell vaccine.
